(19) United States
(12) Patent Application Publication
(10) Pub. No.: US 2023/0233757 A1
(43) Pub. Date: Jul. 27, 2023

(54) METHODS AND SYSTEMS FOR PRESSURE-BASED FLUID CONTROL IN A FLUID DELIVERY SYSTEM

(71) Applicant: INSULET CORPORATION, Acton, MA (US)

(72) Inventor: David NAZZARO, Groveland, MA (US)

(21) Appl. No.: 18/157,394

(22) Filed: Jan. 20, 2023

Related U.S. Application Data
(60) Provisional application No. 63/302,269, filed on Jan. 24, 2022.

(51) Int. Cl. A61M 5/142; A61M 39/12

(52) U.S. Cl. CPC A61M 5/14244; A61M 39/12; A61M 5/16804

(57) ABSTRACT

Fluid delivery devices with passive or pressure-based control valves are described. For example, a fluid delivery device may include a fluid path, a pressure source fluidically coupled to a fluid source storing a fluid, and a pressure-based control valve arranged in the fluid path and configured to move in an opening direction in response to a fluid delivery pressure applied by the pressure source in an upstream portion of the fluid path against the pressure-based control valve, in which the pressure control valve is in an open configuration responsive to the fluid delivery pressure being equal to or greater than a cracking pressure. In some embodiments, the pressure-based control valve may be or may include a bourdon tube. Other embodiments are described.

### RELATED APPLICATIONS

This application claims the benefit of U.S. Provisional Patent Application No. 63/302,269, filed Jan. 24, 2022, the contents of which are incorporated herein by reference in their entirety.

### FIELD OF THE INVENTION

The present disclosure generally relates to a drug delivery system, and, in particular, improved methods and systems for controlling fluid flow in a drug delivery system.

### BACKGROUND

Healthcare providers may prescribe patients wearable fluid delivery devices for delivering fluids, such as liquid medicaments, as part of a treatment regimen. Non-limiting examples of medicaments may include chemotherapy drugs, hormones (for instance, insulin), pain relief medications, and other types of liquid-based drugs. Medicament delivery devices require fine dosage control on a micro-scale. Conventional systems use regulation systems, such as valves, that have accuracy and repeatability challenges with typical dosage levels. For example, existing fluid regulation systems for wearable fluid delivery devices may be negatively impacted by environmental conditions, such as temperature. In addition, existing fluid regulation systems need to be designed to limit the need for constant actuation, for example, to switch valves, due to energy/battery limitations.

It is with considerations of these and other challenges in mind that improvements such as disclosed in the present disclosure may be useful.

### BRIEF DESCRIPTION OF THE DRAWINGS

FIG. 1 illustrates a first exemplary embodiment of an operating environment in accordance with the present disclosure;

FIG. 2 illustrates an embodiment of a wearable fluid delivery device in accordance with the present disclosure

FIG. 3 illustrates a first example flow control device in accordance with the present disclosure; and

FIG. 4 illustrates a second example flow control device in accordance with the present disclosure; and

FIG. 5 illustrates a functional block diagram of an example system suitable for implementing the example processes and techniques in accordance with the present disclosure.

The drawings are not necessarily to scale. The drawings are merely representations, not intended to portray specific parameters of the disclosure. The drawings are intended to depict example embodiments of the disclosure, and therefore should not be considered as limiting in scope. In the drawings, like numbering represents like elements.

### DETAILED DESCRIPTION

The described technology generally relates to techniques and systems for fluid flow control in fluid delivery devices. In general, a fluid flow control assembly, device, valve, and/or the like may be configured to provide passive, pressure-based control of fluid flow for a fluid delivery device. The fluid flow control assembly may be configured to control fluid flow and dosage volume to a patient. In various embodiments, the fluid flow control assembly may be or may include a passive valve configuration that allows flow at a specific, calibrated pressure. In exemplary embodiments, the fluid flow control assembly may be or may include a bourdon tube.

In some embodiments, the fluid flow control assembly may be configured to allow for the flow or release of a set volume of a fluid based on a pressure (for instance, a “cracking pressure”) being exerted on a portion of the fluid flow control assembly. When the pressure in or on the fluid flow control assembly is below the cracking pressure (for instance, the minimum pressure required to “crack” or open the system and allow for fluid flow), the fluid flow control assembly will be closed and will prevent fluid flow. When the pressure in the fluid flow control assembly is above the cracking pressure, a portion of the fluid flow control assembly will be opened to allow for fluid flow, for instance, out of the fluid delivery device and into a patient. In some embodiments, the amount of fluid flow may be determined by the amount of applied pressure within a fluid delivery pressure range (for example, starting at a minimum value to allow for a minimum dosage up to a maximum value to allow for a maximum dosage).

In various embodiments, the fluid flow control assembly may be used within a wearable fluid delivery device for delivering a fluid to a patient. In some embodiments, the fluid may be or may include a medicament. The wearable fluid delivery device may include a reservoir (or fluid source) for holding the fluid, a fluid path in fluid communication with the reservoir, a needle (and/or cannula) in fluid communication with the fluid path to deliver the fluid to the patient wearing the wearable fluid delivery device, and a fluid delivery pump configured to force the fluid from the reservoir, through the fluid delivery path, and into the patient via the needle. In some embodiments, the fluid flow control assembly may be arranged in the fluid delivery path, for example, before the needle, to control the flow of fluid out of the fluid delivery path, into the needle, and into the patient. In various embodiments, operation of the fluid pump may generate pressure (a “fluid delivery pressure”) in the fluid delivery path that is incident on the fluid flow control assembly. The fluid delivery pressure may be the pressure that operates the fluid flow control assembly. For example, if the fluid delivery pressure results in a pressure on the fluid flow control assembly that is greater than the cracking pressure, the fluid flow control assembly may open and allow the flow of fluid into the needle and into the patient.

A fluid control system according to various embodiments may provide multiple technological advantages over conventional systems. In one non-limiting technological advantage, embodiments may provide for the effective, repeatable, and accurate control of fluid flow (and, therefore, medicament dosages) for wearable medicament delivery devices. In another non-limiting technological advantage, embodiments may provide a fluid flow control assembly or valve that is not susceptible (or less susceptible) to environmental conditions, such as temperature. In an additional non-limiting technological advantage, embodiments may provide a fluid flow control assembly or valve that can be dialed in and/or calibrated to delivery specific fluid volumes for specific cracking pressures. In a further non-limiting technological advantage, embodiments may provide a fluid flow control assembly or valve that operates as a check valve, for instance, to prevent backflow and/or syphoning to/from the patient. In a further non-limiting technological advantage, embodiments may provide a fluid flow control assembly or valve that limits or eliminates the need for additional devices (such as switch valves) that require power and other resources that degrade device efficiency and lifespan. In a further non-limiting technological advantage, embodiments may provide a fluid flow control assembly or valve that has suitable tolerances to control cracking pressure and having manufacturing repeatability.

Other embodiments and technological advantages are contemplated in the present disclosure.

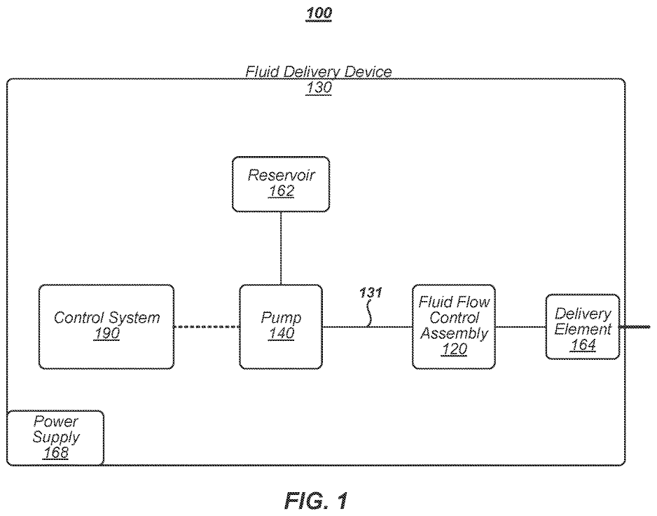

FIG. 1 illustrates an example of an operating environment 100 that may be representative of some embodiments. As shown in FIG. 1, operating environment 100 may include a fluid delivery device 130. In various embodiments, fluid delivery device 130 may include a control system 190 that, in some embodiments, may be communicatively coupled to a computing device (not shown). Computing device may be or may include one or more logic devices, including, without limitation, a server computer, a client computing device, a personal computer (PC), a workstation, a laptop, a notebook computer, a smart phone, a tablet computing device, a personal diabetes management (PDM) device, a microcontroller (MCU) and/or the like.

In some embodiments, computing device may be in wired or wireless communication with fluid delivery device 130, for instance, via control system 190. For example, control system 190 may communicate via various wireless protocols, including, without limitation, Wi-Fi (i.e., IEEE 802.11), radio frequency (RF), Bluetooth™, Zigbee™, near field communication (NFC), Medical Implantable Communications Service (MICS), and/or the like. In another example, control system 190 may communicate via various wired protocols, including, without limitation, universal serial bus (USB), Lightning, serial, and/or the like. Although control system 190 is depicted as being within fluid delivery device 130, embodiments are not so limited. For example, in some embodiments, control system 190 and fluid delivery device 130 may be separate devices. In another example, some or all of the components of control system 190 may be included in fluid delivery device 130. For example, control system 190 may include processor circuitry, a memory unit, and/or the like. In some embodiments, each of control device 190 and fluid delivery device 130 may include a separate processor circuitry, memory unit, and/or the like capable of facilitating occlusion management processes according to some embodiments, either individually or in operative combination. Embodiments are not limited in this context.

Fluid delivery device 130 may be or may include a wearable automatic fluid delivery device directly coupled to a patient, for example, directly attached to the skin of the patient via an adhesive and/or other attachment component. In some embodiments, fluid delivery device 130 may be or may include a medicament delivery device configured to deliver a liquid medicament, drug, therapeutic agent, or other medical fluid to a patient. Non-limiting examples of medicaments may include insulin, glucagon or a glucagon-like peptide, pain relief drugs, hormones, blood pressure medicines, morphine, methadone, chemotherapy drugs, proteins, antibodies, and/or the like.

In some embodiments, fluid delivery device 130 may be or may include an automatic insulin delivery (AID) device configured to deliver insulin (and/or other medication) to a patient. For example, fluid delivery device 160 may be or may include a device the same or similar to an OmniPod® device or system provided by Insulet Corporation of Acton, Mass., United States, for example, as described in U.S. Pat. Nos. 7,303,549; 7, 137,964; and/or 6,740,059, the contents of each of which is incorporated herein by reference in its entirety. Although an AID device and insulin are used in examples in the present disclosure, embodiments are not so limited, as fluid delivery device 130 may be or may include a device capable of storing and delivering any fluid including, without limitation, therapeutic agent, drug, medicine, hormone, protein, antibody, and/or the like.

Fluid delivery device 130 may include a delivery system having a number of components to facilitate automated delivery of a fluid to a patient, including, without limitation, a reservoir 162 for storing the fluid, a pump 140 for transferring the fluid from reservoir 162 and through a fluid path or conduit 131, and into the body of a patient via at least one delivery element 164, such as a needle and/or cannula, configured to be inserted into the skin of the patient. Embodiments are not limited in this context, for example, as delivery system 162 may include more or less components.

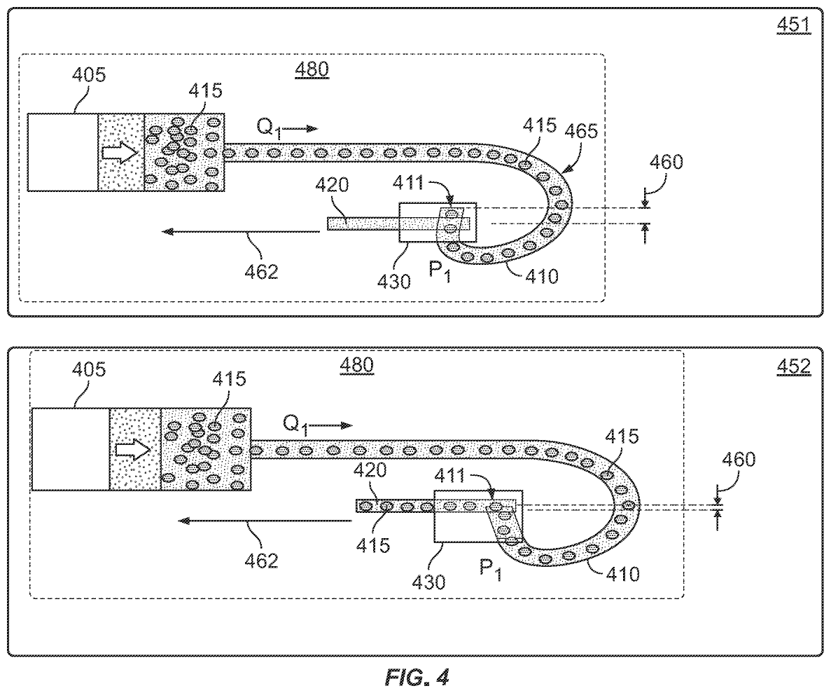

In some embodiments, fluid delivery device 130 may include a fluid flow control assembly 120 configured to control the flow of fluid from in fluid path 131 and out through delivery element 164. In various embodiments, fluid flow control assembly 120 may be a passive, pressure-based control element or assembly. For example, fluid flow control assembly 120 may operate to facilitate the flow of fluid based on a pressure exerted on at least a portion of fluid flow control assembly 120. In various embodiments, fluid flow control assembly may be or may include a bourdon tube (see, for example, FIG. 4).

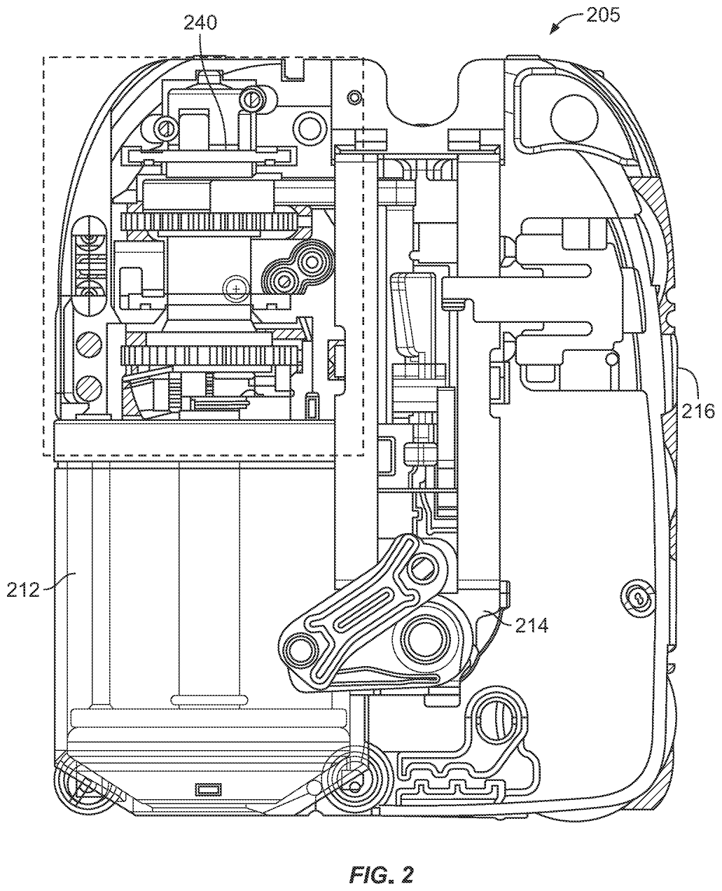

FIG. 2 illustrates an exemplary wearable fluid delivery device in accordance with the present disclosure. In particular, FIG. 4 depicts a top-down view of a wearable fluid delivery device 205. As shown in FIG. 2 a wearable fluid delivery device 205 may include multiple systems to store and delivery a fluid to a patient. In some embodiments, wearable fluid delivery device 205 may include a pump 240. In exemplary embodiments, wearable fluid delivery device 405 may include a reservoir 212 for storing a fluid. Reservoir 212 may be in fluid communication with pump 240 for delivering the fluid to a patient via a needle 214. In some embodiments, components of pump 205 may be arranged within one or more housings 216.

Although a shuttle pump is illustrated in FIG. 2, embodiments are not so limited, as any type of pump capable of operating according to some embodiments is contemplated in the present disclosure. Non-limiting examples of pumps may include positive displacement, syringe-style, reciprocating (diaphragm, piston, and/or the like), MEMS, piezoelectric, and/or the like. Embodiments are not limited in this context.

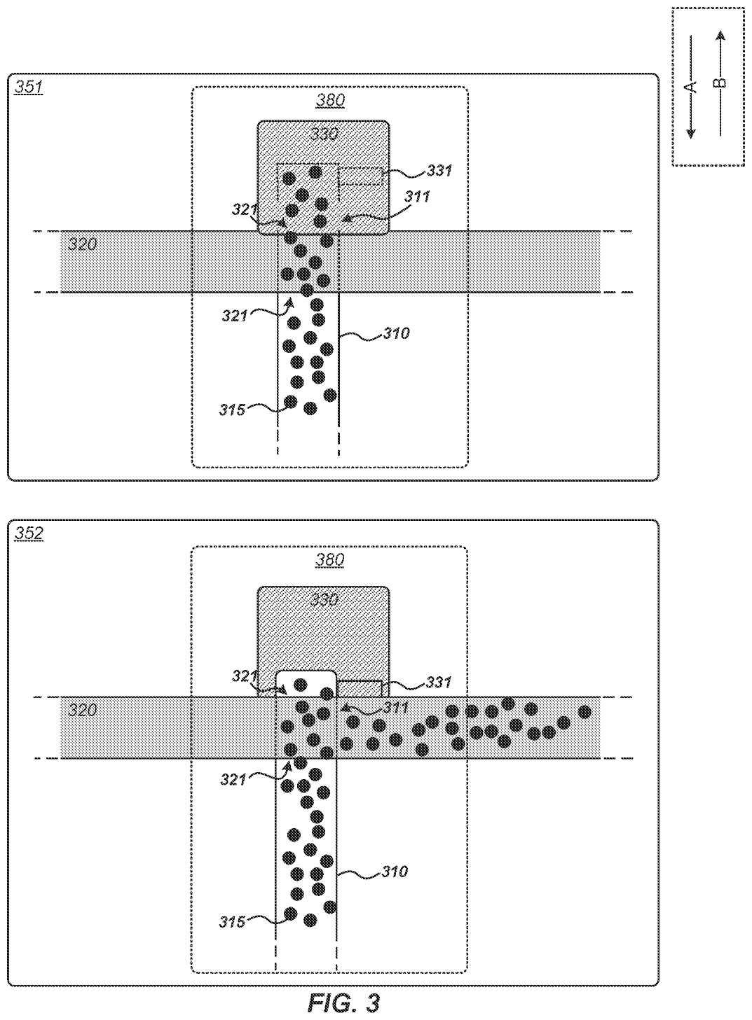

FIG. 3 illustrates a first example flow control device in accordance with the present disclosure. As shown in FIG. 3, a fluid flow control assembly 380 may include a passive or pressure-based control valve or tube 310 configured to move within a fluid path 320. For example, an opening 321 may be arranged in fluid path 320 that allows for control valve 310 to move in one of direction A or B. Opening 321 and fluid path 320 may be hermetically sealed to prevent movement of fluid outside of control valve 310 and fluid path 320. Control valve 310 may have an opening 311 that may allow for fluid to flow out of control valve.

In a “closed” configuration 351, opening 311 may be hermetically sealed by enclosure 330. In various embodiments, enclosure 330 may be formed of a sealing material, including, without limitation, an elastomer, rubber, silicon, a polymer, and/or the like. In closed configuration 351, the fluid delivery pressure on control valve 310 and/or on a fluid 315 within control valve 310 is below a cracking pressure. In some embodiments, when the fluid delivery pressure is below the cracking pressure, control valve 310 may be sealed or dead-headed within enclosure 330. In some embodiments, fluid 315 may include the fluid delivered by the fluid delivery device (for example, retained within control valve 310).

The cracking pressure may be any value or range capable of operating a control valve 310 according to some embodiments. Non-limiting examples of cracking pressures may include about 0.1 psi, about 0.25 psi, about 0.5 psi, about 1 psi, about 2 psi, about 5 psi, about 10 psi, about 100 psi, and any value or range between any two of these values (including endpoints).

In some embodiments, a fluid delivery pressure may be incident on fluid 315 and/or a portion of control valve 310 that is above a cracking pressure. For example, a control system may activate a fluid pump, piston, and/or the like which may cause fluid to flow down a fluid path and contact at least a portion of fluid 315 and/or control valve 310 and generate a fluid delivery pressure (for instance, the pressure of the fluid as pumped by the pump against control valve 310).

When the fluid delivery pressure is greater or equal to a cracking pressure, control valve may move, for example, in direction B (an opening direction) to the “open” configuration 352. In open configuration 352, opening 311 may be moved within fluid path 320, allowing fluid 315 to flow into fluid path 320 (and, for example, out of a needle fluidically coupled to fluid path 320 and into the patient). Although control valve 310 is shown as moving in direction B (opening direction) to enter the open configuration 352, this is for illustrative purposes only as embodiments are not so limited. For instance, flow control valve 310 could move in direction A (or any other direction) to go from the closed configuration 351 to the open configuration 352.

In one non-limiting example, control valve 310 may be arranged on an exit side of a fluid delivery pump, for instance, as an “exhaust” valve, for a fluid-filled chamber. In other embodiments, control valve 310 may be arranged at any point along a fluid path that may be contacted by the fluid delivery pressure. Embodiments are not limited in this context.

In various embodiments, a stop, catch, or other element 331 may be associated with control valve 310. In some embodiments, stop 331 may prevent movement of control valve 310, for example, beyond a certain point within fluid path 320. For instance, if the fluid delivery pressure goes over a threshold pressure, stop 331 may prevent control valve 310 from moving beyond a certain point or distance.

FIG. 4 illustrates a second example flow control device in accordance with the present disclosure. As shown in FIG. 4, a fluid flow control assembly 480 may include a control valve 410 that includes or is essentially in the form of a bourdon tube. In general, a bourdon tube is or includes a flexible or semi-flexible C-shaped tube that may have a fluid arranged therein. When the pressure of the fluid in the C-shaped tube increases, the end of the tube opens out, thereby providing pressure-based displacement of the C-shaped tube.

Although a C-shaped bourdon tube is depicted in some examples, embodiments are not so limited, as a bourdon tube may be configured in a different shape, such as a coil, a helix, a spiral, and/or the like. Embodiments are not limited in this context.

A pressure source 405, such as a pump, piston, or fluid, may exert pressure on fluid 415. The pressure will increase and as a result, the thin-walled bourdon tube, commonly constructed of various metals, will want to displace and straighten. This phenomenon is based on the cross sectional shape of the tube or “moment of inertia” and length/shape of the tube. Accordingly, the cane or hook portion 465 of the bourdon tube 410 will want to straighten. The bourdon tube may be formed of various materials, such as metals (for instance, stainless steel, phosphor bronze, and/or the like).

In the closed state 451, an opening or exit 411 in the end of bourdon tube 410 may be enclosed by sealing element 430. In some embodiments, sealing element 430 may include an elastomeric material. When no pressure is in the system or a pressure below a cracking pressure, opening 411 of bourdon tube 410 may be dead-headed into sealing element and it becomes “shut off” In some embodiments, there may be a gap (not shown) between opening 411 and a delivery needle conduit, such as 420. This gap may operate to establish and calibrate cracking pressure.

As the fluid delivery pressure is applied, and the fluid delivery pressure reaches the cracking pressure, fluid flow control assembly 480 may enter the “open” state 452. In the open state 452, bourdon tube 410 will displace, thus exposing opening 411 to the exit flow channel or conduit 420 (for instance, to delivery needle). For example, bourdon tube 410 may uncoil until opening 411 is in fluid communication with conduit 420. As shown in FIG. 4, distance 460 between opening 411 and conduit 420 may decrease in the open state 452 due to the straightening effect of bourdon tube 410. Flow of fluid 415 into conduit 420 may cause the fluid delivery pressure to decrease (or “bleed off”). Once the fluid delivery pressure drops below the cracking pressure, fluid flow control assembly may return to the closed state 451. For example, as pressure is reduced, bourdon tube 410 may wind back up partially to its naturally-coiled state, thereby cutting off fluid flow (closed state 451).

In some embodiments, the fluid delivery pressure may be controlled such that opening 411 may be maintained (or “float”) within conduit 420 to maintain fluid flow. For example, an initial fluid delivery pressure may operate to crack or open bourdon tube 410 to initiate fluid flow. The flow of fluid may cause the fluid delivery pressure to decrease. Pressure source 405 may be configured to increase the fluid delivery pressure to maintain the fluid flow pressure within a range in which bourdon tube is in the open state 452.

The following Poiuselle's law may be used to illustrate that flow as controlled by fluid flow control assembly 480 may be, at least in part, a function of pressure in the system and overcoming the stiffness or resistance to movement of bourdon tube 410:

$$Q = \frac{π {Pr}^{4}}{8 η l} ,$$

where Q=flow rate, P=pressure, r=radius, n=fluid viscosity, and l=length of tubing. In some embodiments, bourdon tube 410 may be controlled to provide a dosage of a fluid based on Q of Poiuselle's law. In some embodiments, bourdon tube 410 may be configured to provide a fluid, such as insulin, based on pulses, such as 0.5 μL, 5 units of insulin per 100 pulses, and/or the like.

Accordingly, in some embodiments, bourdon tube 410 may operate as a pressure regulator that could be tailored to control a dose. For example, different pressures may cause opening 411 to be opened/closed to a different degree to allow for more/less fluid flow. In this manner, bourdon tube 410 may function as a dosing mechanism responsive to different pressures. For example, a pump or other pressure source could be calibrated with bourdon tube to provide for a known flow or dosage of fluid at specified fluid delivery pressures. For example, a fluid delivery pressure of X may be a cracking pressure, fluid delivery pressure Y may cause bourdon tube 410 to uncoil such that opening 411 allows for a dosage (or dosage rate) of t, fluid delivery pressure Z may cause bourdon tube 410 to uncoil such that opening 411 allows for a dosage of u, and so on.

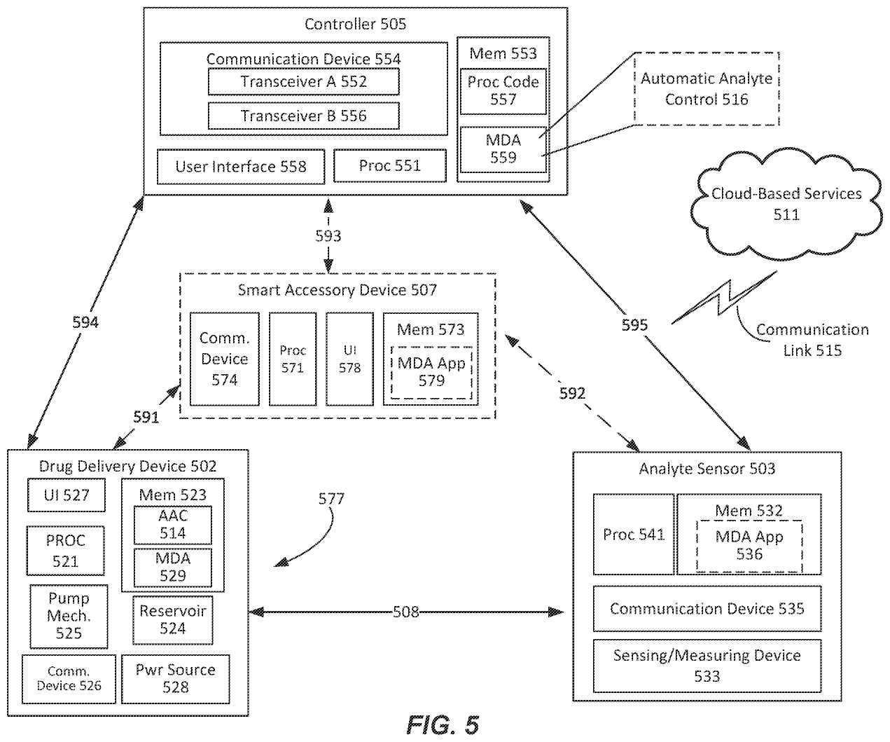

FIG. 5 illustrates a functional block diagram of a system example suitable for implementing the example processes and techniques described herein.

The operating environment 500 may be or may include an automatic drug delivery system that may include components such as an automatic drug delivery system that is configured to determine a drug dosage and deliver the dosage of the drug without any user interaction, or in some examples, limited user interaction, such as in response to a user depressing a button to indicate measurement of blood glucose or another analyte, or the like. “Drug delivery system environment” may refer to a computing and sensing environment that includes cloud based services, a drug delivery system (that may include a controller, a drug delivery device, and an analyte sensor) and optionally additional devices. The components of the drug delivery system environment may cooperate to provide present analyte measurement values or at least accurate estimates of present analyte measurement values to facilitate calculation of optimal drug dosages for a user.

The automatic drug delivery system 500 may implement (and/or provide functionality for) a medication delivery algorithm, such as an artificial pancreas (AP) application, to govern or control automated delivery of a drug or medication, such as insulin, to a user (e.g., to maintain euglycemia—a normal level of glucose in the blood). The drug delivery system 500 may be an automated drug delivery system that may include a wearable automatic drug delivery device 502, an analyte sensor 503, and a management device (for instance, a PDM, smart phone, table computing device, and/or the like) 505.

The system 500, in an optional example, may also include a smart accessory device 507, such as a smartwatch, a personal assistant device or the like, which may communicate with the other components of system 500 via either a wired or wireless communication links 591-593.

The management device 505 may be a computing device such as a smart phone, a tablet, a personal diabetes management device, a dedicated diabetes therapy management device, or the like. In an example, the management device (PDM) 505 may include a processor 551, a management device memory 553, a user interface 558, and a communication device 554. The management device 505 may contain analog and/or digital circuitry that may be implemented as a processor 551 for executing processes based on programming code stored in the management device memory 553, such as the medication delivery algorithm or application (MDA) 559, to manage a user's blood glucose levels and for controlling the delivery of the drug, medication, or therapeutic agent to the user as well as other functions, such as calculating carbohydrate-compensation dosage, a correction bolus dosage and the like as discussed above. The management device 505 may be used to program, adjust settings, and/or control operation of the wearable automatic drug delivery device 502 and/or the analyte sensor 503 as well as the optional smart accessory device 507.

The processor 551 may also be configured to execute programming code stored in the management device memory 553, such as the MDA 559. The MDA 559 may be a computer application that is operable to deliver a drug based on information received from the analyte sensor 503, the cloud-based services 511 and/or the management device 505 or optional smart accessory device 507. The memory 553 may also store programming code to, for example, operate the user interface 558 (e.g., a touchscreen device, a camera or the like), the communication device 554 and the like. The processor 551 when executing the MDA 559 may be configured to implement indications and notifications related to meal ingestion, blood glucose measurements, and the like. The user interface 558 may be under the control of the processor 551 and be configured to present a graphical user interface.

In a specific example, when the MDA 559 is an artificial pancreas (AP) application, the processor 551 is also configured to execute a diabetes treatment plan (which may be stored in a memory) that is managed by the MDA 559 stored in memory 553. In addition to the functions mentioned above, when the MDA 559 is an AP application, it may further provide functionality to enable the processor 551 to determine a carbohydrate-compensation dosage, a correction bolus dosage and determine a basal dosage according to a diabetes treatment plan. In addition, as an AP application, the MDA 559 provides functionality to enable the processor 551 to output signals to the wearable automatic drug delivery device 502 to deliver dosages according to some embodiments.

The communication device 554 may include one or more transceivers such as Transceiver A 552 and Transceiver B 556 and receivers or transmitters that operate according to one or more radio-frequency protocols. In the example, the transceivers 552 and 556 may be a cellular transceiver and a Bluetooth® transceiver, respectively. For example, the communication device 554 may include a transceiver 552 or 556 configured to receive and transmit signals containing information usable by the MDA 559.

The wearable automatic drug delivery device 502, in the example system 500, may include a user interface 527, a controller 521, a drive mechanism 525, a communication device 526, a memory 523, a power source/energy harvesting circuit 528, device sensors 584, and a reservoir 524. The wearable automatic drug delivery device 502 may be configured to perform and execute the processes described in the examples of the present disclosure without input from the management device 505 or the optional smart accessory device 507. As explained in more detail, the controller 521 may be operable, for example, to determine an amount of insulin delivered, JOB, insulin remaining, and the like. The controller 521 alone may determine an amount of insulin delivered, JOB, insulin remaining, and the like, such as control insulin delivery, based on an input from the analyte sensor 504.

The memory 523 may store programming code executable by the controller 521. The programming code, for example, may enable the controller 521 to control expelling insulin from the reservoir 524 and control the administering of doses of medication based on signals from the MDA 529 or, external devices, if the MDA 529 is configured to implement the external control signals.

The reservoir 524 may be configured to store drugs, medications or therapeutic agents suitable for automated delivery, such as insulin, morphine, blood pressure medicines, chemotherapy drugs, or the like.

The device sensors 584 may include one or more of a pressure sensor, a power sensor, or the like that are communicatively coupled to the controller 521 and provide various signals. For example, the pressure sensor may be coupled to or integral with a needle/cannula insertion component (which may be part of the drive mechanism 525) or the like. In an example, the controller 521 or a processor, such as 551, may be operable to determine that a rate of drug infusion based on the indication of the fluid pressure. The rate of drug infusion may be compared to an infusion rate threshold, and the comparison result may be usable in determining an amount of insulin onboard (JOB) or a total daily insulin (TDI) amount.

In an example, the wearable automatic drug delivery device 502 includes a communication device 526, which may be a receiver, a transmitter, or a transceiver that operates according to one or more radio-frequency protocols, such as Bluetooth, Wi-Fi, a near-field communication standard, a cellular standard, or the like. The controller 521 may, for example, communicate with a personal diabetes management device 505 and an analyte sensor 503 via the communication device 526.

The wearable automatic drug delivery device 502 may be attached to the body of a user, such as a patient or diabetic, at an attachment location and may deliver any therapeutic agent, including any drug or medicine, such as insulin or the like, to a user at or around the attachment location. A surface of the wearable automatic drug delivery device 502 may include an adhesive to facilitate attachment to the skin of a user as described in earlier examples.

The wearable automatic drug delivery device 502 may, for example, include a reservoir 524 for storing the drug (such as insulin), a needle or cannula (not shown in this example) for delivering the drug into the body of the user (which may be done subcutaneously, intraperitoneally, or intravenously), and a drive mechanism 525 for transferring the drug from the reservoir 524 through a needle or cannula and into the user. The drive mechanism 525 may be fluidly coupled to reservoir 524, and communicatively coupled to the controller 521.

The wearable automatic drug delivery device 502 may further include a power source 528, such as a battery, a piezoelectric device, other forms of energy harvesting devices, or the like, for supplying electrical power to the drive mechanism 525 and/or other components (such as the controller 521, memory 523, and the communication device 526) of the wearable automatic drug delivery device 502.

In some examples, the wearable automatic drug delivery device 502 and/or the management device 505 may include a user interface 558, respectively, such as a keypad, a touchscreen display, levers, light-emitting diodes, buttons on a housing of the management device 505, a microphone, a camera, a speaker, a display, or the like, that is configured to allow a user to enter information and allow the management device 505 to output information for presentation to the user (e.g., alarm signals or the like). The user interface 558 may provide inputs, such as a voice input, a gesture (e.g., hand or facial) input to a camera, swipes to a touchscreen, or the like, to processor 551 which the programming code interprets.

When configured to communicate to an external device, such as the PDM 505 or the analyte sensor 504, the wearable automatic drug delivery device 502 may receive signals over the wired or wireless link 594 from the management device (PDM) 505 or 508 from the analyte sensor 504. The controller 521 of the wearable automatic drug delivery device 502 may receive and process the signals from the respective external devices as described with reference to the examples of the present disclosure as well as implementing delivery of a drug to the user according to a diabetes treatment plan or other drug delivery regimen.

The analyte sensor 503 may include a controller 531, a memory 532, a sensing/measuring device 533, a user interface 537, a power source/energy harvesting circuitry 534, and a communication device 535. The analyte sensor 503 may be communicatively coupled to the processor 551 of the management device 505 or controller 521 of the wearable automatic drug delivery device 502. The memory 532 may be configured to store information and programming code, such as an instance of the MDA 536.

The analyte sensor 503 may be configured to detect multiple different analytes, such as lactate, ketones, uric acid, sodium, potassium, alcohol levels or the like, and output results of the detections, such as measurement values or the like. The analyte sensor 503 may, in an example, be configured to measure a blood glucose value at a predetermined time interval, such as every 5 minutes, every cycle, or the like. The communication device 535 of analyte sensor 503 may have circuitry that operates as a transceiver for communicating the measured blood glucose values to the management device 505 over a wireless link 595 or with wearable automatic drug delivery device 502 over the wireless communication link 508. While called an analyte sensor 503, the sensing/measuring device 533 of the analyte sensor 503 may include one or more additional sensing elements, such as a glucose measurement element a heart rate monitor, a pressure sensor, or the like. The controller 531 may include discrete, specialized logic and/or components, an application-specific integrated circuit, a microcontroller or processor that executes software instructions, firmware, programming instructions stored in memory (such as memory 532), or any combination thereof.

Similar to the controller 521, the controller 531 of the analyte sensor 503 may be operable to perform many functions. For example, the controller 531 may be configured by the programming code stored in the memory 532 to manage the collection and analysis of data detected the sensing and measuring device 533.

Although the analyte sensor 503 is depicted in FIG. 5 as separate from the wearable automatic drug delivery device 502, in various examples, the analyte sensor 503 and wearable automatic drug delivery device 502 may be incorporated into the same unit. That is, in various examples, the sensor 503 may be a part of the wearable automatic drug delivery device 502 and contained within the same housing of the wearable automatic drug delivery device 502 (e.g., the sensor 503 or, only the sensing/measuring device 533 and memory storing related programming code may be positioned within or integrated into, or into one or more components, such as the memory 523, of the wearable automatic drug delivery device 502). In such an example configuration, the controller 521 may be able to implement the process examples of present disclosure alone without any external inputs from the management device 505, the cloud-based services 511, another sensor (not shown), the optional smart accessory device 507, or the like.

The communication link 515 that couples the cloud-based services 511 to the respective devices 502, 503, 505 or 507 of system 500 may be a cellular link, a Wi-Fi link, a Bluetooth link, or a combination thereof. Services provided by cloud-based services 511 may include data storage that stores anonymized data, such as blood glucose measurement values, historical IOB or TDI, prior carbohydrate-compensation dosage, and other forms of data. In addition, the cloud-based services 511 may process the anonymized data from multiple users to provide generalized information related to TDI, insulin sensitivity, IOB and the like.

The wireless communication links 508, 591, 592, 593, 594 and 595 may be any type of wireless link operating using known wireless communication standards or proprietary standards. As an example, the wireless communication links 508, 591, 592, 593, 594 and 595 may provide communication links based on Bluetooth®, Zigbee®, Wi-Fi, a near-field communication standard, a cellular standard, or any other wireless protocol via the respective communication devices 554, 574, 526 and 535.

Software related implementations of the techniques described herein may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein

1. A fluid delivery device, comprising:
a fluid path;
a pressure source fluidically coupled to a fluid source storing a fluid; and
a pressure-based control valve arranged in the fluid path and configured to move in an opening direction in response to a fluid delivery pressure applied by the pressure source in an upstream portion of the fluid path against the pressure-based control valve,
wherein the pressure control valve is in an open state responsive to the fluid delivery pressure being equal to or greater than a cracking pressure.

2. The fluid delivery device of claim 1, the fluid delivery device comprising a wearable insulin pump.

3. The fluid delivery device of claim 1, the pressure source comprising a fluid delivery pump having a form of at least one of a positive displacement pump, a syringe-style pump, a reciprocating pump, a MEMS pump, or a piezoelectric pump.

4. The fluid delivery device of claim 1, comprising a sealing element to seal an opening in the pressure-based control valve when the fluid delivery pressure is below the cracking pressure.

5. The fluid delivery device of claim 1, the pressure-based control valve comprising a stop element configured to prevent movement of the pressure-based control valve in the opening direction beyond a specified distance.

6. The fluid delivery device of claim 1, wherein the pressure-based control valve is in a closed position when the fluid delivery pressure is below the cracking pressure.

7. The fluid delivery device of claim 1, comprising a control system to control the fluid delivery pressure to cause the pressure-based flow control valve to inject a dosage of the fluid into a patient.

8. The fluid delivery device of claim 1, the pressure-based control valve comprising a bourdon tube.

9. The fluid delivery device of claim 8, the bourdon tube comprising a C-shaped tube configured to straighten in response to the cracking pressure to enter the open state.

10. The fluid delivery device of claim 8, comprising a control system to control the fluid delivery pressure to cause the pressure-based flow control valve to inject a dosage of the fluid into a patient based, at least in part, on a flow rate determined based on Poiuselle's law.

11. A fluid delivery method, comprising:
providing fluid delivery device comprising:
a fluid path,
a pressure source fluidically coupled to a fluid source storing a fluid, and
a pressure-based control valve arranged in the fluid path and configured to move in an opening direction in response to a fluid delivery pressure applied by the pressure source in an upstream portion of the fluid path against the pressure-based control valve, and
controlling the fluid delivery pressure via the pressure source to place the pressure control valve in an open state responsive to the fluid delivery pressure being equal to or greater than a cracking pressure.

12. The method of claim 11, the fluid delivery device comprising a wearable insulin pump.

13. The method of claim 11, the pressure source comprising a fluid delivery pump having a form of at least one of a positive displacement pump, a syringe-style pump, a reciprocating pump, a MEMS pump, or a piezoelectric pump.

14. The method of claim 11, comprising providing a sealing element to seal an opening in the pressure-based control valve when the fluid delivery pressure is below the cracking pressure.

15. The method of claim 11, the pressure-based control valve comprising a stop element configured to prevent movement of the pressure-based control valve in the opening direction beyond a specified distance.

16. The method of claim 11, wherein the pressure-based control valve is in a closed position when the fluid delivery pressure is below the cracking pressure.

17. The method of claim 11, comprising providing a control system to control the fluid delivery pressure to cause the pressure-based flow control valve to inject a dosage of the fluid into a patient.

18. The method of claim 11, the pressure-based control valve comprising a bourdon tube.

19. The method of claim 18, the bourdon tube comprising a C-shaped tube configured to straighten in response to the cracking pressure to enter the open state.

20. The method of claim 18, comprising providing a control system to control the fluid delivery pressure to cause the pressure-based flow control valve to inject a dosage of the fluid into a patient based, at least in part, on a flow rate determined based on Poiuselle's law.
